(19) United States
(12) Patent Application Publication
(10) Pub. No.: US 2023/0220371 A1
(43) Pub. Date: Jul. 13, 2023

(54) METHOD AND DEVICE FOR INACTIVATING VIRUS

(71) Applicant: Noriyasu IKEDA, Kawasaki-shi (JP)

(72) Inventor: Noriyasu IKEDA, Kawasaki-shi (JP)

(21) Appl. No.: 17/923,863

(22) PCT Filed: Mar. 9, 2021
(86) PCT No.: PCT/JP2021/009281

§ 371 Date: Nov. 7, 2022

(30) Foreign Application Priority Data
May 8, 2020 (JP) 2020-082384

(51) Int. Cl. C12N 13/00; A61L 2/08

(52) U.S. Cl. CPC C12N 13/00; A61L 2/08

(57) ABSTRACT

A method and a device that enable inactivation of a virus even if the nature and the like of the virus is unknown. The method for inactivating a virus involves preparing a laser that emits laser light having a specific wavelength, and irradiating a virus with the laser light to inactivate the virus.

### TECHNICAL FIELD

This invention relates to a method and a device for viral inactivation and, in particular, to a method and a device for inactivating novel coronaviruses (COVID-19).

### BACKGROUND TECHNOLOGY

A pandemic situation caused by novel coronaviruses began at the end of 2019. As of May 2020, the pandemic situation has spread worldwide and shows no signs of abating. In our country, a state of emergency has been declared nationwide, and people are being required to refrain from even going outside houses.

In our country, the number of people infected with the novel coronaviruses (COVID-19) at the end of April was reported to be less than 100 per day. However, the number of infected people and the number of deaths are reported on every TV channel from morning to night, and this is giving people a sense of insecurity because they do not know how long the outbreak will continue.

In our country, the conditions for taking the PCR test to check for COVID-19 infection are very strict. Therefore, people who are asymptomatic without fever, cough, etc., who are not considered to be infected cannot undergo PCR testing. Therefore, the number of people infected with the novel coronaviruses (COVID-19) cannot be accurately determined.

Vaccines against the novel coronaviruses are currently being developed in the United States, Germany, China, and other countries, but it will take at least one more year before they are put into practical use. Various medicines for the novel coronaviruses are also being developed. However, the development of vaccines and medicines is time-consuming and costly, and it will take a considerable amount of time before they become available to the general public at a reasonable price.

On the other hand, according to the recently published non-patent literature 1, a new report informs that the novel coronaviruses can be reduced in number by half in 2 minutes at a humidity of 80% and at a temperature of 21-24° C. when they are exposed to sunlight.

### PRIOR ART LITERATURE(S)

### Non-Patent Literature(s)

Non-Patent Literature 1: Internet Article published on Apr. 24, 2020, by Jiji Press, Ltd. (URL: https://www.jiji.com/jc/article?k=2020042400741&g=int)

### SUMMARY OF THE INVENTION

### Problem to be Solved by the Invention

The first object of the present invention is to provide a method and a device that can quickly and inexpensively inactivate coronaviruses.

The second object of the present invention is to provide a method and a device for physically inactivating coronaviruses by taking advantage of their weak points, instead of overcoming coronaviruses with chemical methods such as vaccines, drugs, or the like.

The third object of the present invention is to provide a method and a device that can be effectively applied to passivate coronaviruses even if the viruses mutate.

### Means to Solve the Problem

According to the first aspect of the present invention, there is obtained a method for viral inactivation, characterized by preparing a laser that emits a laser beam having a specific wavelength and irradiating viruses with the laser beam to inactivate the viruses.

According to the second aspect of the present invention, there is obtained the method for viral inactivation described in the first aspect, characterized in that the specific wavelength includes a wavelength in the ultraviolet region.

According to the third aspect of the present invention, there is obtained the method for viral inactivation described in the first or the second aspect, characterized in that the laser beam is emitted in an atmosphere with a humidity of 80% or more and with a temperature of 20° C. or more.

According to the fourth aspect of the present invention, there is obtained the method for viral inactivation described in any one of the first through the third aspects, characterized in that the viruses are novel coronaviruses (COVID-19).

According to the fifth aspect of the present invention, there is obtained a device for viral inactivation, that includes the laser used in the method for viral inactivation described in any one of the first through the fourth aspects, and that is adapted to switch at least one of an output power, a wavelength, and an irradiation time according to the nature of novel coronaviruses (COVID-19).

According to the sixth aspect of the present invention, there is obtained the device for viral inactivation described in the fifth aspect, characterized by including the laser which comprises an emitting part body that emits the laser beam, means for varying at least one of the output power, the wavelength, and the irradiation time of the laser beam, a waveguide that guides the laser beam from the emitting part body, and an emitting end for irradiating an object with the laser beam from the waveguide.

### Effect of the Invention

According to this invention, viruses can be inactivated by selecting at least one of the output power, the wavelength, and the irradiation time of the laser beam according to natures of the viruses. Here, inactivation of the viruses means that the growth of the viruses by activation can be suppressed. Since commercially available ultraviolet laser systems, etc., can be used as lasers for viral inactivation, this invention can suppress viral growth at a very low cost compared to developing vaccines and medicines. In addition, the possibility of mutating viruses can be dealt with by selecting the output power, the wavelength, and the irradiation time of the laser beam.

### BRIEF DESCRIPTION OF THE DRAWING

FIG. 1 shows an example of a laser used in a viral inactivation method according to the present invention.

### MODE FOR EMBODYING THE INVENTION

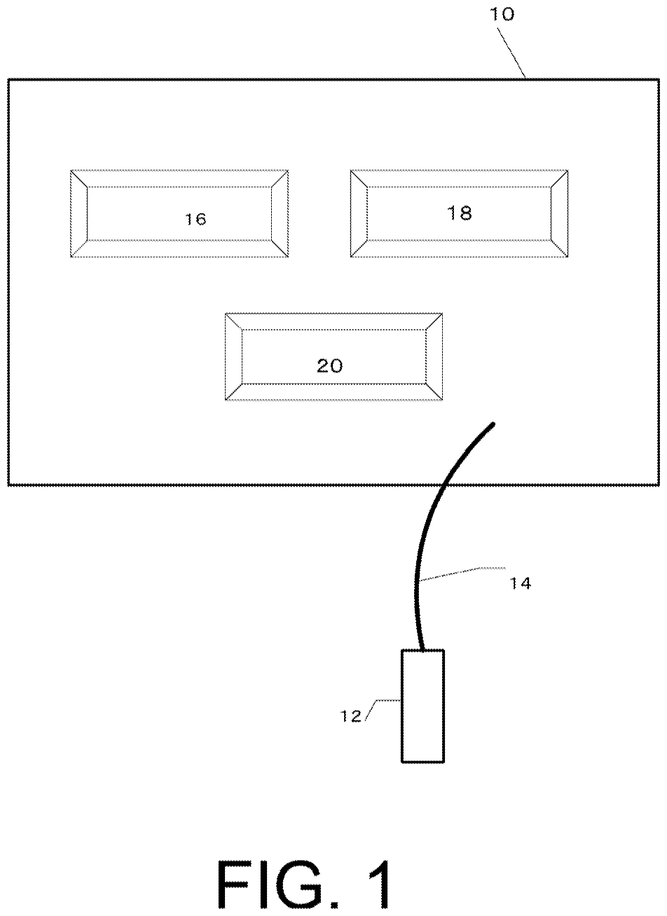

FIG. 1 is a conceptual diagram showing an example of a laser used in a viral inactivation method according to the present invention.

### Example 1

The illustrated laser comprises a laser body (emitting part body) 10 that emits a laser beam, an emitting part 12 that irradiates an object with the laser beam emitted from the laser body 10, and a waveguide 14 that leads the laser beam from the laser body 10 to the emitting part 12, and a waveguide 14 that guides the laser beam from the laser body 10 to the emitting part 12. Furthermore, on the front side of the laser body 10, there are a power control unit 16 that can vary the output power of the laser beam, a wavelength control unit 18 that can vary the wavelength of the laser beam, and a time control unit 20 that controls the irradiation time. The power control unit 16, the wavelength control unit 18, and the time control unit 20 may be adjusted manually or remotely using a remote controller.

In the laser body 10, laser elements, such as semiconductor lasers, YAG lasers, a drive circuit, a power supply circuit, and other components are integrated.

The power control unit 16 can adjust the output power of the laser beam within a range of power that does not affect the object itself, e.g., within a range of 1 mW or less. Desirably, the wavelength control unit 18 is able to control the wavelength variably within a range of wavelengths shorter than 375 nm. However, the wavelength control unit 18 may variably control the wavelength from the wavelengths in the ultraviolet region to the wavelengths in the visible light or the infrared region. Furthermore, the time control unit 20 is a timer that sets the irradiation time of the laser beam.

When the laser beam irradiates a living object, such as a human body, the emitting end 12 may have a shape that can be inserted into the living body. For example, the emitting end 12 may have a structure having a rod-like shape that can be inserted into a throat or bronchus. When an inactivation test is performed outside the living body, viruses as the object contained in a container such as a petri dish may be irradiated with the laser beam. By the test, it is possible to optimize the wavelength, the output power, and the irradiation time according to the viruses as the object.

When actually using the viral inactivation method of the present invention, the object containing the viruses is placed in an atmosphere with a temperature of 20° C. or higher, preferably 35° C. or higher, and a humidity of 50% or more, preferably 80% or more.

In this state, the emitting part 12 is directed toward the object, and the laser beam is emitted. In this case, a laser beam having a specific wavelength selected according to the nature of the viruses to be irradiated is emitted from the laser body 10 through the waveguide 14.

When the nature of the viruses is unknown, the output power, the wavelength, and the irradiation time of the laser beam are varied according to the viruses by trial and error to determine the optimum conditions. The optimum conditions can be determined by varying at least one of the output power, the wavelength, and the irradiation time according to the nature of the viruses, so that the conditions necessary for inactivation of the viruses are derived.

If the nature of the viruses is already known, the laser beam with the output power and the wavelength selected for the viruses is emitted to the object for a predetermined time. This optimizes the output power, the wavelength, and the irradiation time of the laser beam required to inactivate the viruses.

Once the optimum conditions, such as the output power, the wavelength, and the irradiation time are found out, according to the present invention, activated viruses can be inactivated or neutralized by irradiating the object with the laser beam under the optimum conditions.

### INDUSTRIAL APPLICABILITY

This invention is very economically advantageous because unknown viruses can be neutralized or inactivated by physically irradiating the viruses without relying on vaccines, medicines, or other means. This invention is also significant in view of using the laser against the viruses.

### DESCRIPTION OF REFERENCE NUMERALS

10 Laser body
12 Emitting part
14 Waveguide
16 Power control unit
18 Wavelength control unit
20 Timer (time control unit)

1. A method for viral inactivation, characterized by preparing a laser that emits a laser beam having a specific wavelength and irradiating viruses with the laser beam to inactivate the viruses.

2. The method for viral inactivation according to claim 1, wherein the specific wavelength includes a wavelength in the ultraviolet region.

3. The method for viral inactivation according to claim 1, wherein the laser beam is emitted in an atmosphere with a humidity of 80% or more and with a temperature of 20° C. or more.

4. The method for viral inactivation according to claim 1, wherein the viruses are novel coronaviruses (COVID-19).

5. A device for viral inactivation, including the laser used in the method for viral inactivation described in any one of the first through the fourth aspects, and adapted to switch at least one of an output power, a wavelength, and an irradiation time according to the nature of novel coronaviruses (COVID-19).

6. The device for viral inactivation according to claim 5, including the laser which comprises an emitting part body that emits the laser beam, means for varying the output power of the laser beam, means for varying the wavelength of the laser beam, means for varying the irradiation time of the laser beam, a waveguide that guides the laser beam from the emitting part body, and an emitting end for irradiating an object with the laser beam from the waveguide.
